(19) United States
(12) Patent Application Publication
(10) Pub. No.: US 2016/0367342 A1
(43) Pub. Date: Dec. 22, 2016

(54) System and Method of Installing Dental Prostheses for Passively Influencing Tongue Position to Reduce Airway Obstruction

(71) Applicants: R. Michael Alvarez, Fremont, CA (US); J. Sergio Alvarez, Newark, CA (US)

(72) Inventors: R. Michael Alvarez, Fremont, CA (US); J. Sergio Alvarez, Newark, CA (US)

(21) Appl. No.: 14/742,807

(22) Filed: Jun. 18, 2015

(51) Int. Cl. A61C 8/00; A61F 5/56

(52) U.S. Cl. CPC A61C 8/0083; A61F 5/566; A61C 8/0022; A61C 8/0074; A61C 8/0062; A61F 2230/0071; A61F 2230/0076

(57) ABSTRACT

A system and method of installing dental implants to treat obstructed airflow via passive repositioning of the tongue. The present method includes the steps of installing one or more implants between the teeth along the mucogingival junction, such that the implants extend into the oral cavity and make contact with the undersurface of the tongue when the tongue is in a resting position. The positioning of the implants causes them to gently irritate the undersurface of the tongue, innervating the lingual nerve. Innervation of the lingual nerve causes the genioglossus to contract and repositions the tongue to a more anterior and superior position as compared to its resting position, vacating the airway and dilating the pharynx to improve airflow therethrough. Both the positioning and the size of the implants are customizable, thereby ensuring proper contact between the implants and the tongue regardless of topographical differences of patients' oral cavities.

### FIELD OF THE INVENTION

The present invention relates to dental prostheses. More specifically, the present invention relates to a method of installing dental prostheses having protuberances at specific locations within a patient's mouth. The protuberances rest against the base of the tongue, innervating the nerves within the tongue and thereby passively causing the tongue to reposition itself to a more anterior and superior position within the mouth. The repositioning of the tongue reduces the amount of airway blockage caused by the tongue as compared to its normal resting position.

### BACKGROUND OF THE INVENTION

Obstructive sleep apnea (OSA) is a disorder in which a sleeper's breathing is interrupted by the whole or partial blockage of the pharyngeal airway. During pharyngeal constriction, breathing may be shallow or may cease all together. Abnormal breathing can last for ten seconds to several minutes. Patients may snore, make choking noises, grind their teeth or have small spasms as they try to breathe. Over time, untreated OSA can lead to additional health problems, including bruxism, heart disease, high blood pressure, stroke, memory loss, and other forms of brain damage. Treatments for obstructive sleep apnea include oral dental devices that open up the throat to allow improved air flow, machines with oxygen masks that force air into the throat (continuous positive airway pressure), and lifestyle changes including dietary changes and exercise.

Decreased muscle tone in the throat is the primary cause of OSA in most otherwise healthy patients. Decreased muscle tone creates two major issues that can result in the complete or partial blockage of the upper airway: constriction of the pharyngeal opening and migration of the tongue into the path of the airway. Decreased muscle tone causes the pharyngeal opening to constrict because when the muscles controlling the pharyngeal opening are less tone, they are more prone to relax, thereby reducing the diameter of the pharyngeal opening and thus reducing the rate that air can flow therethrough. Decreased muscle tone causes the tongue to migrate for much the same reason. The less tone the muscles of the tongue are, the more likely the tongue is to move from its natural resting position to a more relaxed posterior position that obstructs the flow of air through the upper airway.

Oral dental devices or some mechanical support implants currently used in the treatment of OSA are designed to prevent airway closure by forcing the mandible forward and maintaining an open airway. This mandibular repositioning maintains the pharynx passageway in an open state, permitting continuous airflow through the passageways of the throat. While these oral dental devices may be helpful to some patients, they physically reposition the mandible without requiring any active participation by the patient's orofacial muscles. Proper muscle tone and strengthening of the orofacial muscles could reduce frequency of apnea episodes and increase the pharynx's ability to maintain dilation. However, because there is no active participation by the patient's orofacial muscles with the use of current mechanical support implants, the muscles are not toned or strengthened, forcing the patient to use the mechanical implants in perpetuity.

Flexing tongue musculature can manipulate the size of the opening of the pharyngeal airway and maintain the tongue in a position in which it does not block the airway, obviating the need for mechanical movement of the mandible. The genioglossus, the exterior muscle of the tongue responsible for tongue protrusion and depression, is an important muscle for controlling both the constriction of the pharyngeal opening and the positioning of the tongue. When in flexion, the genioglossus moves the tongue forward, maintaining the tongue in a position out of the path of the airway, and also causes the muscles controlling the pharyngeal opening to dilate.

Pharyngeal dilation can also be achieved via stimulated natural tongue protrusion without mechanical repositioning of the mandible. Motor innervation for all tongue muscles comes from the hypoglossal nerve (CN XII), except for the palatoglossus, which is supplied by the pharyngeal plexus (vagus nerve). Tongue motor function is voluntary and not regularly susceptible to reflex responses. Sensory experience of the anterior (front two-thirds) of the tongue is supplied by a branch of the trigeminal nerve (CN V₃) known as the lingual nerve. The hypoglossal nerve and lingual nerve are in communication along the anterior border of the hypoglossal muscle. Thus, stimulation of the lingual nerve across different portions of the tongue surface can be translated to the hypoglossal nerve and affect voluntary motor function of the extrinsic muscle structures.

Dental tori, which are naturally occurring growths or accumulations of bone cells of the surfaces of the oral cavity, can trigger a reflex response in the tongue by impinging upon the various motor nerves of the tongue. This repositioning of the tongue in response to dental tori can in turn manipulate the size of the pharynx and increase the size of the airway. However, this is merely a side effect of the presence of the dental tori and it is impracticable to attempt to harness the natural formation of dental tori to influence tongue positioning and airway size for multiple reasons. First, there is no way to precisely control the formation of the dental tori because their growth is not well characterized. Second, although the tori themselves generally do not require treatment, the underlying cause of the dental tori, e.g. bruxism, often has a plethora of undesirable side effects and requires treatment.

Alternatively, electrical stimulation of the genioglossus has also been shown to manipulate pharyngeal cross-section size. Stimulation across the anterior portions of the tongue produces contraction of the genioglossus and forward tongue protrusion, whereas stimulation of posterior areas of the tongue results in bunching of the posterior tongue and constricts the diameter of the pharynx. However, although electrical stimulation is useful in clinical settings, electrical stimulation of a user's oral cavity during sleep or while engaging in athletics would be cumbersome and potentially dangerous. Therefore, there is a need in the prior art for targeted and safe physical stimulation of the tongue to affect dilation of the airways of the throat and improve muscle strength.

Therefore, there is a need in the prior art for a system and method of installation thereof that utilizes projections that resemble dental tori to passively reposition the tongue by triggering a natural reflex response in order to actively recruit tongue muscles in order to treat OSA, thereby obviating the need for treatment options utilizing mechanical support or electrical stimulation.

### SUMMARY OF THE INVENTION

In view of the foregoing disadvantages inherent in the known types of oral implants and methods of repositioning the tongue now present in the prior art, the present invention provides a new method of passively influencing the positioning of the tongue via installing dental prostheses in a patient's mouth that rest against and gently irritate the tongue, thereby innervating the motor nerves of the tongue and causing the tongue to reposition itself in order to increase the size of the opening of the pharyngeal airway.

The present invention comprises a system of dental implants adapted to treat OSA and other such maladies associated with or caused by obstructed airflow and a method of installation thereof. The present method comprises the steps of installing implants into a patient's mouth such that the implants impinge upon the patient's tongue, gently irritating the tongue and triggering a reflex response that causes the tongue to reposition itself in response to the stimuli. The implants comprise at least one lower implant installed into the mandible of the patient and at least one upper implant installed into the upper palatal arch. The one or more lower implants are installed between the teeth of the mandible, along the mucogingival junction, such that the fastening portion of the implant does not make contact with the root of the patient's teeth nor the nerve extending between each of the tips of the roots. The one or more upper implants are installed behind and superior to the incisive papilla on the upper palatal arch.

The lower implants are installed such that they extend horizontally into the oral cavity from the mandible. When the tongue is in its standard resting position, the undersurface of the tongue makes contact with the lower implants projecting from the interior surface of the mandible, generating an unconscious reflexive response that causes the tongue to automatically reposition itself in a more superior and anterior position that clears the airway and provides improved airflow therethrough. The present invention further comprises an upper implant that is installed behind and superior of the incisive papilla along the upper palatal arch. The upper implant assists in guiding the tongue to the improved resting position.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a view of the mandibular portion of a mouth having the present system installed therein.

FIG. 1B shows a view of the maxillary portion of a mouth with the present system installed therein.

FIG. 2 shows an exploded view of an embodiment of an implant of the present system.

FIG. 3 shows a perspective view of the mandibular portion of a mouth having the present system installed therein.

FIG. 4 shows a vertical cross-sectional view of a mouth having the present system installed therein.

FIG. 5A shows a view of the mandibular portion of a mouth having the present system installed therein in an alternative configuration.

FIG. 5B shows a view of the mandibular portion of a mouth having the present system installed therein in an alternative configuration.

### DETAILED DESCRIPTION OF THE INVENTION

Reference is made herein to the attached drawings. Like reference numerals are used throughout the drawings to depict like or similar elements of the system and method of installing dental prostheses for influencing the position of the tongue and thereby reducing airway obstruction. For the purposes of presenting a brief and clear description of the present invention, the preferred embodiment will be discussed as used for treating OSA. The figures are intended for representative purposes only and should not be considered to be limiting in any respect.

The present invention comprises a method of installing a plurality of permanent or semi-permanent dental implants into the upper and lower portions of a patient's mouth in positions that cause the implants to impinge upon one or more of the nerves of the tongue, such as the lingual nerve. As the tongue makes contact with the implants, the nerve or nerves targeted by the present method are innervated by the contact with the implants, triggering an involuntary reflex response that causes the muscles of the tongue to contract. The contraction of the muscles causes the tongue to reposition itself within the mouth, clearing the tongue from the airway and dilating the pharyngeal opening, improving airflow through the airway. The present system thereby passively and effectively treats OSA, strengthening the muscles of the tongue without forced mechanical intervention.

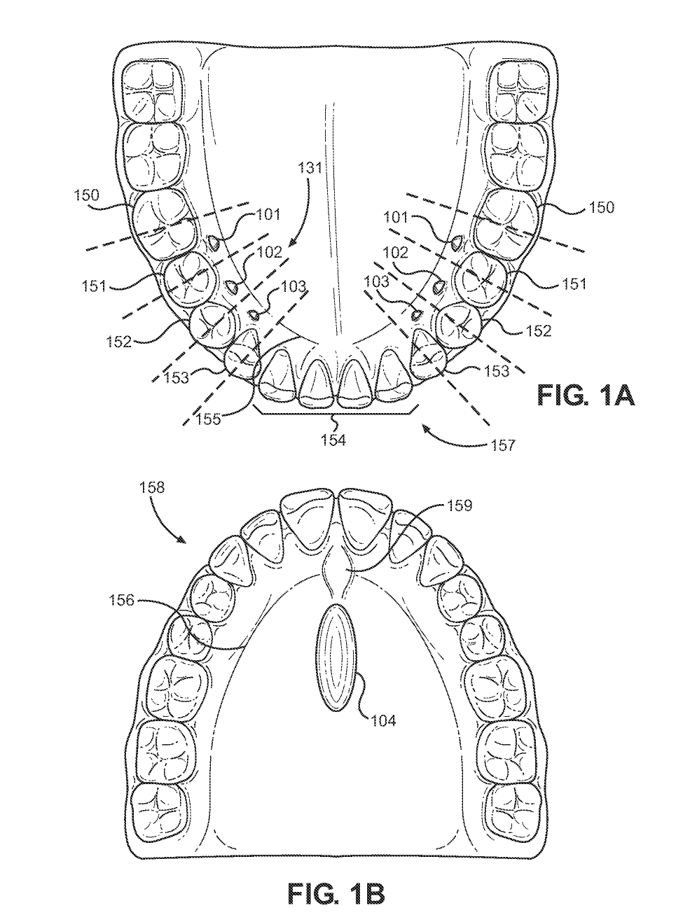

Referring now to FIGS. 1A and 1B, there are shown views of the mandibular and maxillary portions of a mouth having the present system installed therein. The present invention comprises a method of installing a plurality of implants into the upper and lower portions of the mouth of a patient, wherein the implants are arranged in a set of one or more bilateral pairs of lower implants 131 installed into the mandible 157 of the patient and one or more upper implants 104 installed into the upper palatal arch 156 of the patient. The lower implants 131 are installed between the patient's teeth such that the implants do not make contact with the roots of the teeth nor the nerves extending from the tips of the roots. In an illustrative embodiment of the present invention, the bilateral pairs of lower implants 131 comprise a first lower implant 101 installed between the first molar 150 and the second bicuspid 151 along the mucogingival junction 155, a second lower implant 102 installed between the second bicuspid 151 and the first bicuspid 152 along the mucogingival junction 155, and a third lower implant 103 installed between the first bicuspid 152 and the cuspid 153 along the mucogingival junction 155. The lower implants 131 are installed such that they extend horizontally from the mandible 157 into the oral cavity, thereby allowing the lower implants 131 to impinge upon the undersurface of the tongue, gently irritating the tongue when it makes contact with the lower implants 131.

The depicted configuration of the lower implants 131 is intended solely to be illustrative and should not be read as limiting in any way. Although not shown in the referenced figures, the lower implants 131 are installable between any of the teeth, including the incisors 154, in any number and any arrangement. Furthermore, the lower implants 131 need not be arranged such that they occupy successive spaces between the teeth of the mandible.

The one or more upper implants 104 are installed into the upper palatal arch 156 of the maxilla 158. In an illustrative embodiment of the present method, a single upper implant 104 is installed into the upper palatal arch 156, behind and superior to the incisive papilla 159. The upper implant 104 assists in guiding the tongue when it is repositioning. Much as with the lower implants 131, the upper implant 104 influences the positioning of the tongue by gently irritating the surface the tongue when the tongue is pressed thereagainst, thereby triggering a reflex response that causes the tongue to reposition itself out of the path of the airway.

In an illustrative embodiment of the present invention, the exposed portion of the lower implants 131 are semi-spherical in shape and the exposed portion of the upper implant 104 is semi-ellipsoidal in shape. The size of the lower implants 131 and the upper implant 104 are customized to account for differences in the size and shape of patients' mouths, thereby ensuring that the lower implants 131 are positioned to impinge upon the undersurface of the patient's tongue and the upper implant 104 is positioned to impinge upon the upper surface of the patient's tongue. For example, the first and the third lower implants 101, 103 have a diameter of 2 mm to 3 mm, the second lower implant 102 has a diameter of 4 mm, and the base portion of the upper implant 104 is an ellipse having a minor diameter from 2 mm to 4 mm and a major diameter from 0.5 inches to 1.0 inch.

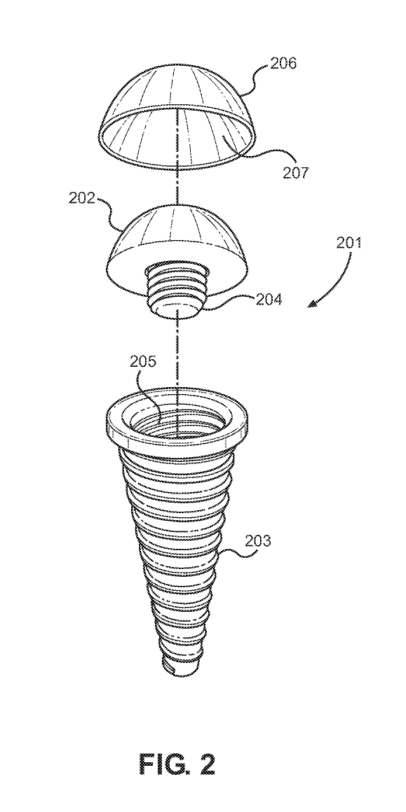

Referring now to FIG. 2, there is shown an exploded view of an embodiment of an implant of the present system. An illustrative embodiment of an implant 201 comprises a cap 202 or abutment that is securable to an abutment screw 203 or fastener via a connector. The removable attachment between the cap 202 and the abutment screw 203 allows for differently sized and shaped caps 202 to be interchangeably secured to the screw 203 in order to customize the installation of the present system for each user. In the depicted embodiment of the present invention, the connector comprises a threaded connector 204 extending from undersurface of the cap 202 that engages with a complementary threaded recess 205 in the screw 203. In an alternative embodiment of the present invention, the connector comprises a snap connection.

In the depicted embodiment of the implant 201, the cap 202 has a semi-spherical shape. However, various embodiments of the implant 201 comprise differently shaped caps 202. For example, one embodiment of the implant comprises a cap 202 having a semi-ellipsoidal shape. When installed into the mouth of a patient, the abutment screw 203 is used to secure the implant 201 into the mandibular bone or the hard palate of the patient and the cap 202 is left exposed, extending into the oral cavity.

The implant 201 further comprises one or more shells 206 that are affixable over the cap 202. In one embodiment, the shells 206 comprise a semi-spherical shape. In another embodiment, the shells comprise a semi-ellipsoidal shape. The shells 206 further comprise a hollow interior having a size and shape corresponding to the size and the shape of the cap 202 or another shell 206, thereby allowing the shells 206 to be placed thereover. The shells 206 are bonded to the cap 202 or each other via an acrylic adhesive, permanently mounting the shells 206 thereto. The shells 206 have a surface area and a thickness, allowing for one or more of the shells 206 to be stacked in order to customize (i) the surface area of the exposed portion of the implant 201 so that it impinges upon a greater surface area of the tongue and (ii) the distance to which the exposed portion of the implant 201 extends into the oral cavity. Because the oral topography of each individual is unique, successive layers of the shells 206 can be bonded to the implant 201 in order to customize each individual implant 201 for each patient in order to accommodate for differences in distances between the interior surface of the oral cavity and the tongue of the patient.

The removable connection between the cap 202 and the abutment screw 203 portions of the implant 201 provides a first degree of customization because differently sized and shaped caps 202 can be interchangeably affixed to the abutment screw 203 once the abutment screw 203 is installed within the mouth of a patient. The ability to bond a successive series of shells 206 over the cap 202 provides a further degree of customization, allowing the size and shape of the exposed portion of the implant 201 to be precisely tailored on a patient-by-patient basis.

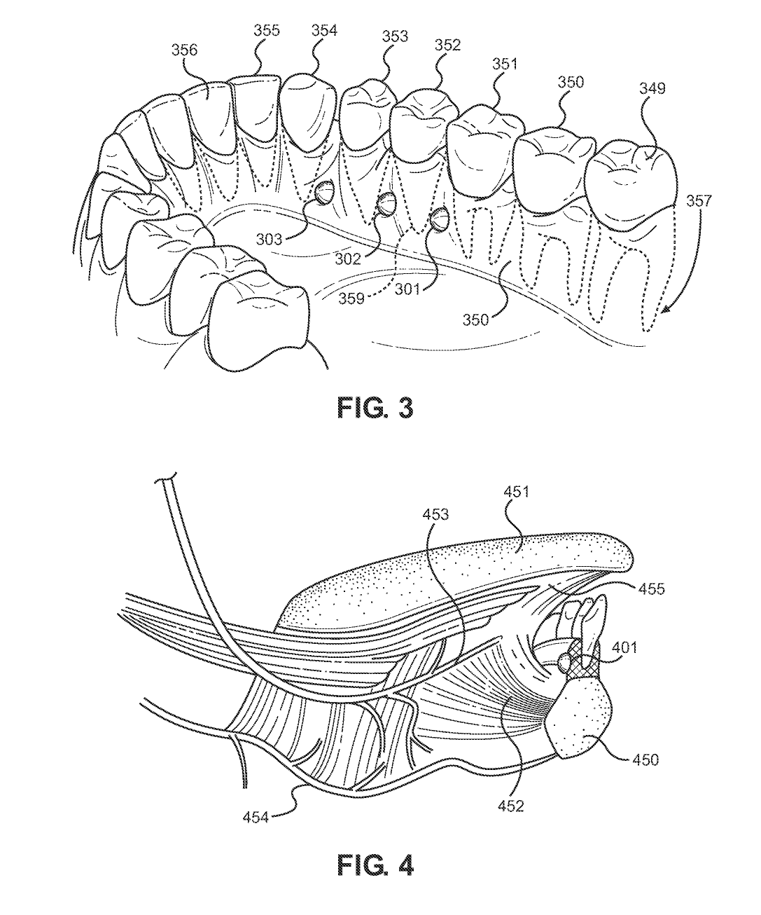

Referring now to FIG. 3, there is shown a perspective view of the mandibular portion of a mouth having the present system installed therein. The lower implants 301, 302, 303 are installed into the mandible 357 of a patient along the mucogingival junction 358, between the roots 359 of the teeth such that the screw or fastener portion of the implants 301, 302, 303 does not make contact with the roots 359, nor the nerves extending from the tips of the roots 359 (not shown).

This arrangement places the implants in a spaced relationship, separated by one or more teeth. In an illustrative embodiment of the present invention, the implants comprise a first implant 301 disposed between the first molar 351 and the second bicuspid 352, a second implant 302 disposed between the second bicuspid 352 and the first bicuspid 353, and a third implant 303 disposed between the first bicuspid 353 and the cuspid 354. However, this configuration for the installation of the implants is not intended to be limiting in any way. The implants are also installable between the other gaps between the teeth, e.g. between the central incisor 356 and the lateral incisor 355, the lateral incisor 355 and the cuspid 354, the first molar 351 and the second molar 350, and the second molar 350 and the third molar 349.

Referring now to FIG. 4, there is shown a vertical cross-sectional view of a mouth having the present system installed therein. The lower implants 401 are installed into the mandible 450, between the teeth 455, such that the implants 401 extend horizontally therefrom into the oral cavity. When in its resting position, the lower implants 401 impinge upon the undersurface 455 of the tongue 451. The impingement of the lower implants 401 on the tongue 451 gently irritates the undersurface 455 thereof, which in turn causes the innervation of the lingual nerve 453. Innervation of the lingual nerve 453 then carries over to the hypoglossal nerve 454, which controls the contraction of the genioglossus 452. When the hypoglossal nerve 454 is innervated, it causes the genioglossus 452 to contract, clearing the tongue 451 from the airway.

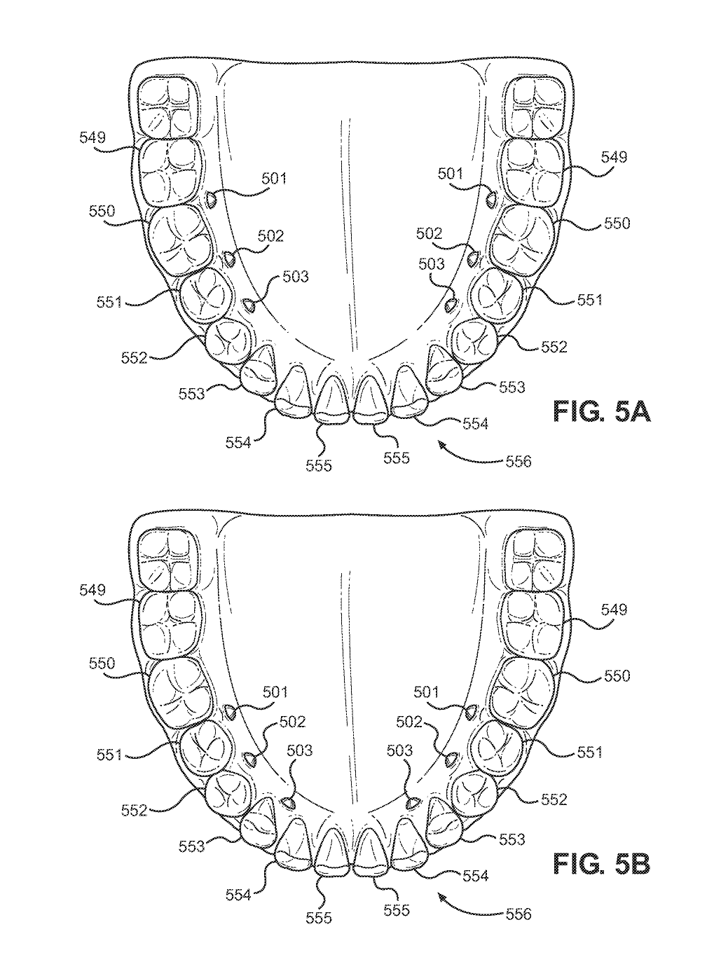

Referring now to FIGS. 5A and 5B, there are shown views of the mandibular portion of a mouth having the present system installed therein in alternative configurations. Because the anatomy of each individual is unique, the present system is installable in a variety of different configurations in order to accommodate differences in the size of teeth, the distances between teeth, the thickness of the gingiva, the size of the tongue, the shape of the tongue, the location of the nerves of the tongue, and other such variables. Therefore, the present system is installable between any of the teeth and the configurations of the dental implants depicted in FIGS. 1, 5A, and 5B are intended solely to be illustrative.

FIG. 5A shows an illustrative configuration for the present system installed into a mandible 556, wherein the first implant 501 is installed between the second molar 549 and the first molar 550, the second implant 502 is installed between the first molar 550 and the second bicuspid 551, and the third implant 503 is installed between the second bicuspid 551 and the first bicuspid 552. FIG. 5B shows an illustrative configuration for the present system installed into a mandible 556, wherein the first implant 501 is installed between the first molar 550 and the second bicuspid 551, the second implant 502 is installed between the second bicuspid 551 and the first bicuspid 552, and the third implant 503 is installed between the cuspid 553 and the lateral incisor 554. In other configurations, the implants can be installed between any of the other gaps in the teeth not shown, e.g. between the lateral incisor 554 and the central incisor 555. The positioning of the implants is completely determined on a case-by-case basis. It should be further noted that although the illustrative configurations of the present system are depicted as having three implants, no claim is made as to a specific number of implants utilized by the present method and system. The positioning of the implants and the number of implants necessary to elicit the desired reflex response of the tongue is dependent upon anatomy of the patient, which creates topographical differences in the oral cavity.

The desired positioning for the implants can be determined experimentally prior to the installation of the abutment screws of the implants into the mouth of the patient by utilizing a mouth guard having projections thereon, wherein the projections correspond to potential installation points for the implants. This allows patients to try different configurations for the projections that elicit the desired reflex response in the tongue. Once this ideal positioning for the projections is achieved, the permanent solution represented by the installation of the implants can then be undertaken. When installing the implants, the locations of the projections on the mouth guard can serve as a guide for the installation of the implants. Once the implants are installed, the implants can be customized by affixing differently sized and shaped interchangeable caps to the abutment screws. The caps can then be further customized by bonding one or more shells to the caps, allowing each individual implant to be tailored for the specific anatomy of the patient.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A method of reducing airway obstruction, the method comprising the steps of:
installing one or more lower implants along a mucogingival junction, between a plurality of teeth such that the one or more lower implants extend horizontally from a mandible and do not make contact with a root of each of the plurality of teeth;
wherein the one or more lower implants comprise an abutment screw portion installable into the mandible and a cap extending into an oral cavity;
installing one or more upper implants behind and superior to an incisive papilla along an upper palatal arch.

2) The method of claim 1 further comprising the steps of:
affixing one or more shells to the cap of at least one the one or more lower implants and the one or more upper implants;
wherein the one or more shells increase at least one of a surface area of the cap and a distance to which the cap extends into the oral cavity.

3) The method of claim 2, wherein the one or more shells are affixed to the cap via acrylic adhesive.

4) The method of claim 1, wherein the cap is interchangeably attached to the abutment screw.

5) The method of claim 4, wherein the cap is attached to the abutment screw by a threaded connection.

6) The method of claim 4, wherein the cap is attached to the abutment screw by a snap connection.

7) The method of claim 1, wherein the one or more lower implants are configured to impinge upon the undersurface of a tongue and innervate the lingual nerve, causing a genioglossus to contract and thereby removing the tongue from an airway.

8) The method of claim 1, wherein the cap of the one or more lower implants comprises a semi-spherical shape.

9) The method of claim 1, wherein the one or more upper implants comprise an abutment screw portion installable into an upper palate and a cap extending into the oral cavity.

10) The method of claim 9, wherein the cap of the one or more upper implants comprises a semi-ellipsoidal shape.

11) The method of claim 9, wherein the cap of the one or more upper implants is interchangeably attached to the abutment screw of the one or more upper implants.

12) A dental implant system for reducing airway obstruction, the system comprising:
one or more lower implants comprising an abutment screw portion installable into a mandible and a cap extending into an oral cavity when the abutment screw is installed into the mandible;
one or more upper implants comprising an abutment screw portion installable into an upper palate and a cap extending into the oral cavity;
one or more shells affixable to the cap of the one or more lower implants and the one or more upper implants.

13) The system of claim 12, wherein the cap of the one or more lower implants is interchangeably attachable to the abutment screw of the one or more lower implants.

14) The system of claim 12, wherein the cap of the one or more upper implants is interchangeably attachable to the abutment screw of the one or more upper implants.

15) The system of claim 12, wherein the one or more lower implants comprise a semi-spherical shape.

16) The system of claim 12, wherein the one or more lower implants comprise a semi-ellipsoidal shape.
